(19) United States
(12) Patent Application Publication
(10) Pub. No.: US 2024/0350053 A1
(43) Pub. Date: Oct. 24, 2024

(54) A DEVICE FOR TRACKING THE MINDFULNESS OF A USER AND A METHOD THEREOF

(71) Applicant: AVANTARI TECHNOLOGIES PRIVATE LIMITED, Hyderabad (IN)

(72) Inventors: Bhairav SHANKAR, Hyderabad (IN); Vimal CHANDRU, Hyderabad (IN); Rajesh CHANDRASHEKAR, Hyderabad (IN); Hari KEERTHIPATI, Sullurupet (IN)

(21) Appl. No.: 18/685,924

(22) PCT Filed: Nov. 22, 2022
(86) PCT No.: PCT/IN2022/051020

§ 371 Date: Feb. 23, 2024

(30) Foreign Application Priority Data
Nov. 22, 2021 (IN) 202141053506

(51) Int. Cl. A61B 5/16; A61B 5/00; A61B 5/0205; A61B 5/024; G16H 40/67

(52) U.S. Cl. CPC A61B 5/165; A61B 5/0205; A61B 5/6826; A61B 5/7278; A61B 5/7445; A61B 5/7455; A61B 5/7475; G16H 40/67; A61B 5/02416

(57) ABSTRACT

The present invention relates to the field of wearable smart devices for monitoring key vital parameters of the user. Specifically, it pertains to a device which effectively calculates the amount of time that the individual is meditating and mindful. The present invention is more specifically pertains to the device which effectively calculates the amount of time that the individual is meditating and mindful regardless of the form of meditation that the user chooses to meditate.

### FIELD OF INVENTION

The present invention relates to a device for monitoring and tracking the mindfulness of a user, and more particularly to a wearable smart device that effectively calculates the amount of time that the individual is meditating and mindful and a method thereof.

### BACKGROUND OF THE INVENTION

It has been well established that an activity like meditation helps with not only relieving stress, but has the ability to slow ageing in the body. The 2009 Nobel Prize winning study in Medicine showed how Telomeres, tiny particles at the end of every chromosome is affected by stress. Telomeres are known to indicate the body's age viz, the longer they are the more healthy and younger our bodies appear, and the shorter they are, we begin to display signs of age as well as become more susceptible to diseases. Studies have shown that meditating for a few short minutes a day has shown to make the telomeres resistant to highly stressful situations.

However, meditation appears to be an esoteric activity, with many confused as to what is actually is. The assumption is that meditation is to be in a state akin to the one we are in when we sleep, and is prevalent due to the visual appearance of meditation: the image of a seated person with their eyes closed brings forth the idea that behind those eyes, the person is resting and may even be asleep.

While certain types of meditation do assist the body in moving towards a relaxed or a parasympathetic state, various schools of meditation believe it to be in active state, one that requires intense concentration on a particular object. This object could be a point on the body, one's own breath, visualization of a ball of light within one's own body or an object in front of the person.

Pickkers, P. (2012). The Influence of Concentration/Meditation on Autonomic Nervous System Activity and the Innate Immune Response A Case Study, Psychosomatic Medicine, 74(5), 489-495-489&ndash-ndash; 494-489-494. describes two experiments in focus-based meditation where meditation evokes the stress, or the sympathetic response from individuals, indicating that meditation which involves concentration is noticed by the human body as a stressed state.

Further studies in the matter, such as Kamath, C. (2019). Evaluation of heart rate dynamics during meditation using Poincaré phase plane symbolic measures. International Journal of Biomedical Engineering and Technology, 29(4), 385-402. have shown that other focus-based meditation in multiple techniques by experts has a pattern of a predominant para-sympathetic state before a meditation and a deep sympathetic state during it.

However, studies such as Nesvold, A., Fagerland, M., Davanger, S., Solberg, E., Holen, A., Sevre, K., & Atar, D. (2011). Increased heart rate variability during nondirective meditation: European Journal of Preventive Cardiology, 19(4), 773-780. show that techniques such as non-directive meditation result in an extended parasympathetic state.

Furthermore studies such as Kanchibhotla, D., Subramanian, S., & Kaushik, B. (2021). Association of yogic breathing with perceived stress and conception of strengths and difficulties in teenagers. Clinical Child Psychology and Psychiatry, 26(2), 406-417. have indicated meditations such as Sudarshan Kriya can be considered to be form of breath meditation.

Hence meditation can exist as a form of relaxation meditation, focus meditation and breathing meditation, which indicates that to know how much time we have spent being mindful requires a form of measurement device which can learn the form of meditation we are doing and begin to measure the parameters associated with it. Accordingly, such device needs to be robust and flexible to have algorithms that can pick up the indices of mindfulness from all three forms of meditation.

US20190254590A1 describes a system that measures heart rate variability to provide biofeedback during a meditation exercise, however it doesn't classify meditations according to the type they are, and treats meditation as simply an activity that is meant to allow users to reach a desired heart rate variability. This prior art fails to show any algorithms and processes to derive the amount of time the individual is mindful for during the meditation exercise. The biofeedback it provides the user, which is purely based on heart rate variability values, do not give any indication to the user on how to improve the form of meditation they are practicing.

US10960174B2 describes a system for monitoring the personal health of an individual by monitoring their autonomic nervous system through a plurality of sensors, and even recommends a single meditation course to help maintain the individual's health. However, this document fails to indicated any information on whether the person is in a meditative state or not, and while it prescribes meditations of various types, i.e. breathing or otherwise, its measurements aren't classified by the basis of the meditation performed.

WO2016119654A1 describes a system for providing a respiratory guide to the individual for a breathing-based meditation or exercise, through a light emitting device. The prior art further describes measuring the effects the meditation has on the autonomic nervous system, however does not provide any information of how the individual performed while they were meditating.

WO2016119664A1 describes a method of deriving the user's respiratory sinus arrhythmia by monitoring the ECG and uses Heart Rate Variability to measure the effect it has on the autonomic nervous system during the breathing training session, however fails to describe any method of deriving the meditative state with the person is in a breathing-based meditation.

While most prior art fail to discuss a method of tracking or measuring the mindful state, prior art do exist for measuring other parameters such a stress levels. KR102185933B1 uses a plurality of sensors to detect the state that the autonomic nervous system is in, and is describes a method to detect the stressed state, however fails to measure the meditative state.

Hence, there is a need for a device that monitors and tracks the time during which a user is mindful and to categorize the type of meditation the user is involving themselves. The present invention provides such a device for tracking the mindfulness of a user during a meditation process, and a method therefor.

### OBJECTIVES OF THE INVENTION

The primary objective of the present invention is to provide a device that calculates the amount of time user is meditating and mindful regardless of the form of meditation that the user choses to meditate.

Another objective of the present invention is to provide a device that allows users to measure their quality of meditation irrespective of the type of meditation viz. breathing, relaxation or the focus type.

Yet another objective of the present invention is to provide a device that can be worn by user throughout a day comfortably while the device can measure all the wholistic parameters of the user including sleep, calorie burn, stress level and blood oxygen.

Yet another objective of the present invention is to provide a device that can notify the users when their stress level increases so that necessary steps can be mitigated accordingly.

Still another objective of the present invention is to provide a smart wearable device that can be charges on the go.

Still another objective of the present invention is to provide a smart wearable device that measures heart rate variability and calculates the amount of time the users have been truly meditating during their session along with the amount of time they have been distracted.

Another objective of the present invention is to develop a device that can worn by the user viz. to be able to easily pass over user's finger joints and accommodate any flexing of his/her hands into fists.

Yet another objective of the present invention is to develop/design a device with a split in its circular form to allow for some degree of flexibility.

Yet another objective of the present invention is to provide a smart device with a unique case for charging the device when required.

Still another objective of the present invention is to provide a smart device that includes a display allowing the user to select type of meditation or activity he/she wish to do by way of tapping or swiping the device thereby negating the need for a phone to be connected to it at all times.

### SUMMARY OF THE INVENTION

According to one of the embodiments, the present invention provides a wearable smart device (100), with an in-built chargeable battery, in contact with a user for tracking the amount of time the user is mindful during a session of meditation comprising an input module allowing the user to select and register at-least one form of meditation session from a list consisting of breathing, relaxation and focus meditation to establish a session for monitoring; a plurality of sensor modules recognising the input provided by the user; a display module displaying the registered type of meditation that will be tracked by the device; a plurality of sensory modules measuring beat-by-beat heart rate of the user; a processing module (101), with an inbuilt wireless communication unit, to calculate the time difference between consecutive heartbeats and converting the same into frequencies in order to determine the mindfulness of the user during the session; and a memory module (108), associated with the processor module, to store the collected and analysed data during the session.

The plurality of sensor modules further includes photo plethysmograph (PPG) sensor in infra-red and red lights (102), 6-axis gyroscope (103), conduction sensory, touch detection sensor (104) and temperature sensor.

According to another embodiment of the present invention, the device is in the form of a ring (303) with a slit (302) that can be easily worn on any finger and further includes a strip of micro holes (502) on its surface letting the RGB LED (105) to shine through its enclosures. The device includes a means of charging through a handy charging dock (200), that is in power line communication (201) with the device (100) to send signal on the charge level of the device.

The charging dock (200) comprises of a battery (201), battery charger (202) and a micro-controller (203) wherein the charging dock (200) opens by way of sliding to reveal the device, and the device is automatically lifted up to make the device easily accessible. The input module includes receiving inputs from the user by way of swiping and/or tapping the surface/face of the device.

According to yet another embodiment, the present invention also provides a method for tracking and determining the time for which the user has been mindful during a session of meditation viz. breathing, focus and relaxation.

According to still another embodiment, the present invention also tracks the sleep, exercise and stress level of the user and indicates/notifies them accordingly.

### BRIEF DESCRIPTION OF THE DRAWINGS

The invention is further described in the detailed description that follows, by reference to the noted drawings by way of illustrative embodiments of the invention, in which like reference numerals represent similar parts throughout the drawings. The invention is not limited to the precise arrangements and illustrative examples shown in the drawings:

FIG. 1 shows the block diagram of the components/modules comprising the device;

FIG. 2 shows the device per the present invention with the kind of interactions it allows by way of touch detection sensors;

FIG. 3 shows how the ring's shape is able to navigate over larger joints (13) on the finger, yet remain snug on the finger when it reaches its final location;

FIG. 4 shows micro holes on the surface of the ring;

FIG. 5 and FIG. 6 illustrates other types of interactions that are allowed with a combination of the touch sensors and the inbuilt inertial measurement unit;

FIG. 7 shows portable carry case according to one of the embodiments of the present invention, which slides to reveal the ring within;

FIG. 8 shows to the lifting mechanism, and FIG. 9 shows the charging of the device through the carry case's internal battery;

FIG. 10 illustrates graphical representation of predominance of frequencies in a person's heart rate in the range between 0.03-0.5 Hz;

FIG. 11 shows flow chart illustrating method of detection and measurement of a user's breathing;

FIG. 12 illustrates graphical representation of the relaxation pattern of a user, and highlights how heart beat appears when the user is relaxed and when they're not;

FIG. 13 shows flow chart illustrating method of calculation of mindful relaxation of the user;

FIG. 14 illustrates graphical representation of the focus, and highlights the changes in the ratio between low frequency and high frequency of a user during a focus meditation; and

FIG. 15 shows flowchart illustrating method of calculation of mindful focus of a user during his/her meditation.

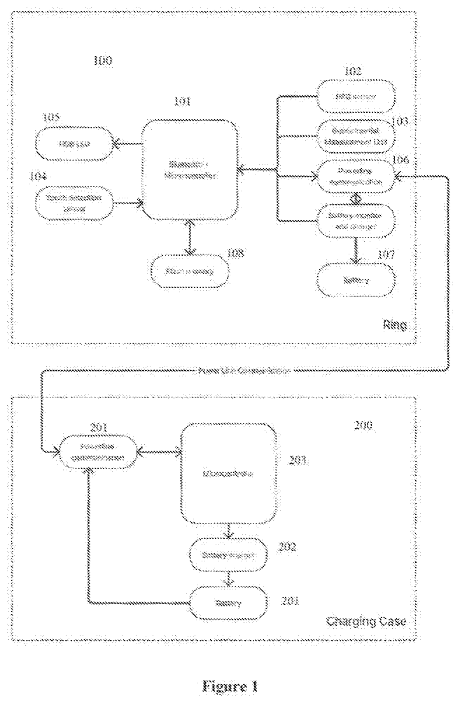

### DETAILED DESCRIPTION OF THE PREFERRED EMBODIMENTS

Reference will now be made in detail to the exemplary embodiment(s) of the present invention. Before describing in detail embodiments that are in accordance with the present disclosure, it should be observed that the embodiments reside primarily in combinations of different components/modules of the device.

In the document, the terms “comprises,” “comprising,” or “including” or any other variation thereof, are intended to cover a non-exclusive inclusion, such that a composition, system, method, article, device or apparatus that comprises a list of elements does not include only those elements but may include other elements not expressly listed or inherent to such compositions, system, method, article, device, or apparatus. An element proceeded by “comprises . . . a” does not, without more constraints, preclude the existence of additional identical elements in the process, product, method, article, device or apparatus that comprises the element.

Any embodiment described herein is not necessarily to be construed as preferred or advantageous over other embodiments. All of the embodiments described in this detailed description are illustrative, and provided to enable persons skilled in the art to make or use the disclosure and not to limit the scope of the disclosure, which is defined by the claims.

It will be apparent to those skilled in the art that various modifications and variations can be made to the present invention without departing from the spirit and scope of the invention. Thus, the present invention covers the modifications and variations of this invention provided they come within the scope of the appended claims and their equivalents.

In the following description, for the purpose of explanation, numerous specific details are set forth in order to provide a thorough understanding of the present invention. It will be apparent, however, to one skilled in the art that the present invention can be practiced without these specific details.

According to one of the embodiments, the present invention provides a wearable smart device with a chargeable case, for tracking and monitoring the amount of time a user wearing it has been mindful during the time he/she spent meditating.

According to another embodiment, the present invention provides for a device that measures key vital parameters of the user including Heart Rate variability, Oxygen Saturation, Galvanic Skin Response, Skin Temperature and motion in 6-axis.

Yet another embodiment of the present invention discloses a smart device that uses a plurality of sensors including such as Galvanic Skin Response, and the LF/HF ratio from heart rate variability to determined the stress levels of the wearer.

Still another embodiment of the present invention provides a smart wearable device that tracks user's sleep, their daily lifestyle, such as exercise, commute and work, and is able to intelligently recognize when there are moments of high stress to the user.

Yet another embodiment of the present invention discloses a contains a wearable device that includes a small display using a range of colours to communicate with the user as well as a small vibrational motor to notify him/her that they are feeling stress and it's time to meditate.

Still another embodiment of the present invention will also help the smart device communicate with a smart phone/device connected with it, wherein such smartphone locates meditations across the internet and recommend the right one to the user depending on their current situation.

Another embodiment of the present invention provides for a wearable device in the form of a ring including a means of charging via a charging dock, which also doubles as the ring's carry case as well as a meditation assist to the user.

Yet another embodiment of the present invention provides for a wearable device that will also inform the user through haptic or visual feedback to do certain activities to allow him to live a holistic life such as exercise, meditate, eat and sleep on time.

According to still another embodiment of the present invention, the sensors on the wearable ring will also be able to determine if the person is having physiological issues such as apnea, arrhythmia and cardiac illnesses.

Accordingly, a smart wearable device (100) per the present invention comprises of a processing module/processor with an inbuilt wireless communication module within (101), and is connected to a plethora of sensors including photo plethysmograph (PPG) sensor (102) in infrared and red lights, 6-axis gyroscope (103) and accelerometers, conduction sensors, touch detection sensors (104), and temperature sensors. The device receives the interactions of a user by means of the touch sensor (104) and communicate to the user through RGB LEDs (105). Through the photo plethysmograph sensors (102), the processing module (101) determines the heart rate variability of the user, which is then analyzed by the processing module (101). It further includes a battery (107) with a battery monitor and charger, and a flash memory (108).

The said device (100) is in power line communication (106) with a charging case/dock (200) that include a battery (201), battery charger (202) and a micro-controller (203). The dock (200) communicates with the device (100) through the power line (201), i.e. it sends signals to the device (100) while on charge. The dock (200) is capable of charging the wearable device (100) independently (i.e. without it being plugged in) as it has its own internal battery (201) within its enclosure, thereby being a mobile convenient charger.

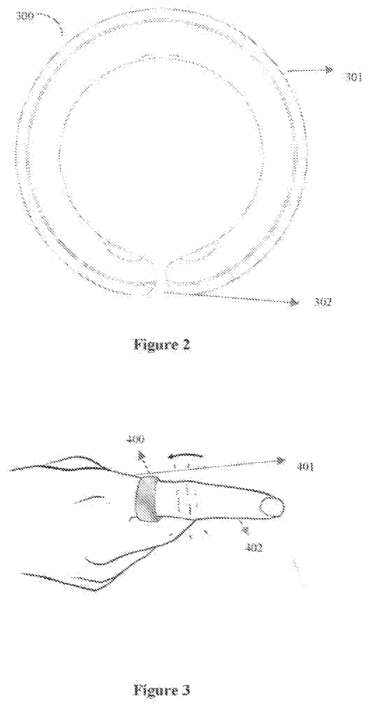

FIG. 2 illustrates shape of the device according to one of the embodiments of the present invention. The device (300) is purposefully designed with a split (302) in its circular ring shape (303). In order to detect heart rate variability (HRV), the sensors need to touch the finger snugly. To avoid sensors digging into the skin using bumps that press into the skin, the ring clasps onto the finger and allows for a degree of flexibility to change its shape to the natural movements of the finger during the day. FIG. 3 shows how the ring's (400) shape is able to navigate over larger joints (401) on the finger (402), yet remain snug on the finger when it reaches its final location.

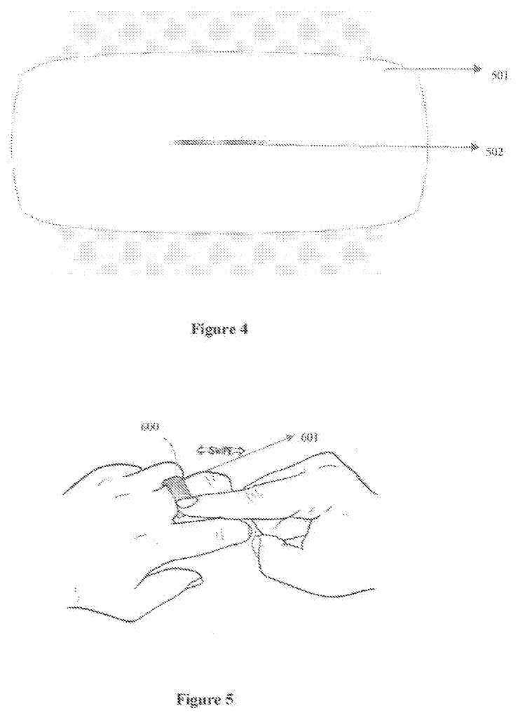

FIG. 4 shows presence of micro holes (502) on the surface of the ring (501) which lets the RGB LED (105) to shine through the metal enclosure. It is to be noted that size of the holes is under 30 microns, which restricts water to enter the enclosure and disturb the electronics, as the water's surface tension would prevent it from passing through the small pores (502).

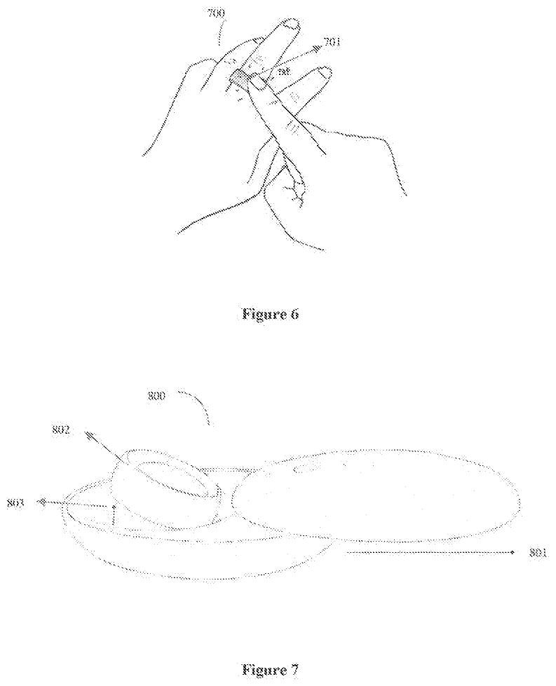

The smart wearable device further includes a display (601) on the face of it, which acts as the principal form of communication between the functioning of the ring (600) and the user. FIG. 5 shows the kind of interactions it allows by way of touch detection sensors (104) such as swiping the surface of the ring. The other types of interactions that are allowed with a combination of the touch sensors (104) and the inbuilt inertial measurement unit (103) to detect the user tapping the ring. The touch sensors (104) are important here to negate any accidental taps on the ring (700), that can occur during an activity or by accident (refer to FIG. 6). When the user taps the ring (700), which would be recognized by the accelerometer and gyroscope, and with every tap, the ring cycles through the three forms of meditation, i.e. breathing, relaxation or focus meditation. The selected meditation will be indicated on the ring through the display (701) on the ring (700).

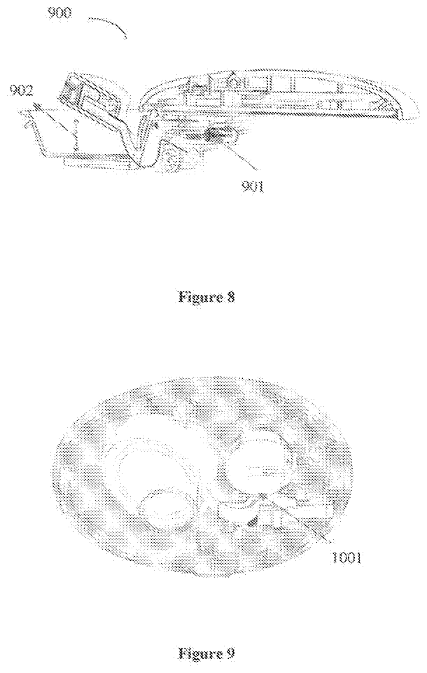

FIG. 7 shows portable carry case (800) according to one of the embodiments of the present invention, which slides (801) to reveal the ring (802) within. The ring automatically is lifted up (803) to make the ring easy to access to the user. FIG. 8 relates to the lifting mechanism and the charging of the device through the carry case's (900) internal battery. The carry case (900) has a micro damper placed within it (901) to ensure the ring doesn't rise up (902) too aggressively. The ring is charged via Pogo pins placed in the carry case (900), which make contact with the charging electrodes on the ring's inner surface. The fit is ensured by magnets that make the ring easy to fall onto the case, while keeping it in place. The carry case's internal battery (1001) enables the dock to be charge the ring without the need for the dock to be plugged into power as shown in FIG. 9.

### Method of Calculation of Mindfulness of the User

According to the swipe/tap of the user, the processor of the smart device registers the form of meditation and then would select the appropriate algorithm to measure the selected form of meditation. If the wearer of the present invention selects breathing form of meditation, the ring begins to record their heart rate variability metrics as they meditate.

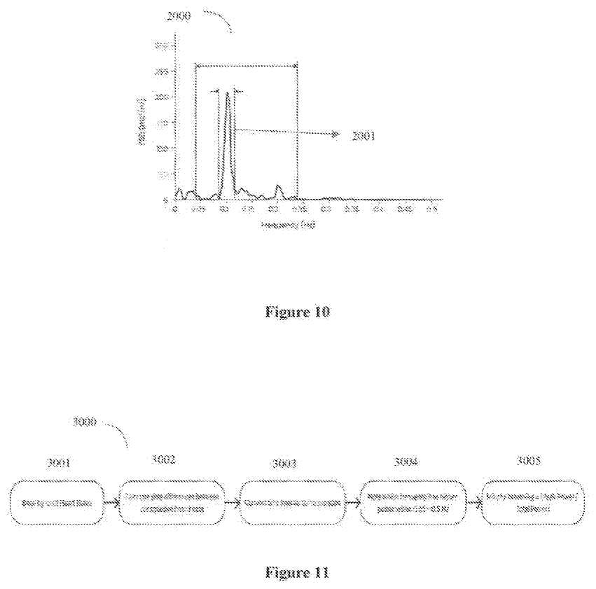

The smart wearable device knows how well the user is breathing and converts a sample of data acquired into its corresponding power spectrum using fast Fourier transform. The user's respiratory sinus arrhythmia will be indicated as a spike in the power spectrum in the low frequency range (0.03-0.5 Hz) corresponding to inhale and exhale lengths from 2 seconds to 33 seconds. FIG. 10 shows graphical representation of predominance of frequencies in a person's heart rate in the range between 0.03-0.5 Hz (2000) wherein it can be noticed that the peak in this signal (2001) is large, and corresponds to the user's breathing rate.

Accordingly, the smart wearable device tracks the beat-by-beat heartrate of a user (3001), and calculates the difference between consecutive heartbeats (3002). The processor/processing module of the device then converts such detected intervals into frequencies (3003), and monitors which frequency has higher power within 0.03 to 0.5 Hz (3004). Thus, the mindful breathing is calculated by dividing the peak power obtained by the total power (3005) as shown in FIG. 11. In other words, as the breathing meditation requires that the user focusses on breathing at a certain rate consistently, the processor of the smart wearable device then selected a region of ±0.05 Hz from the frequency which corresponds to the user's respiratory sinus arrhythmia. This region can be widened or narrowed depending on the user's setting. The processor then integrates all the power within that region, and integrates all the power that is outside the region in the power spectrum. It then estimates that the user is mindfully breathing while within the region, and hence the time spent in mindfulness is related to the integrated power within the frequency range divided by the power that is outside it multiplied by the duration of the sample that was used in the initial analysis.

In yet another embodiment of the present invention, the sample can vary in length depending on the battery capacity of the device, and can be adjusted accordingly to reduce computational time to improve the battery performance of the device. The device then begins to count up this time. When the user taps the ring again to end the meditation practice, the display on the ring indicates the percentage and amount of time the user has been mindful during the session.

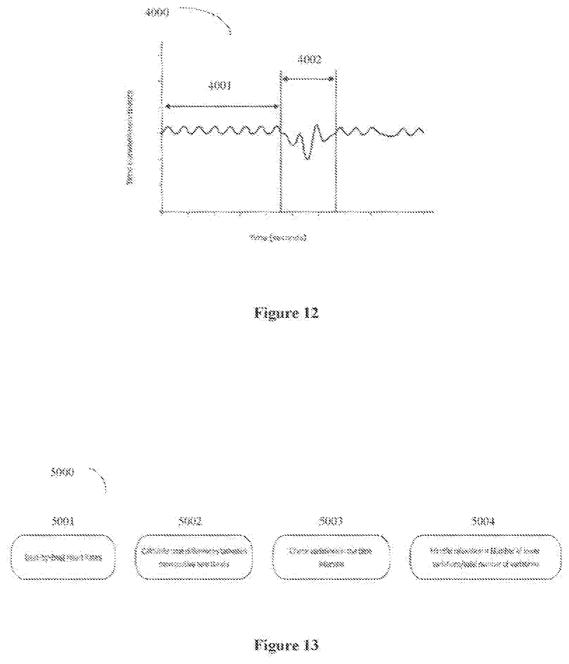

In the even the user selects a relaxation meditation, the processor begins to acquire heart rate variability date from the user. FIG. 12 shows graphical representation of the relaxation pattern of the user (4000), and highlights how heart beat appears when the user is relaxed (4001) and when they are not (4002).

FIG. 13 shows flow chart illustrating method of calculation of mindful relaxation of the user (5000) wherein the device uses the heart rate variability data of the user collected to analyze the amount of time the user has been relaxed using the relaxation algorithm. The processor collects individual heart rate data point (5001) and compares an acquired data value with the one that is immediately preceding it (5002). It then checks the difference between these consecutive points (5003). As relaxation is associated with high frequency variations between consecutive pulses, however, while the change is frequent, the value of time difference is not high. Therefore, the processor of the device sets a threshold for the variations in the differences between consecutive pulses. This threshold is calculated from empirical evidence, and is decreased for the user depending on the user. If the user is able to sustain significant portions of time with lower variations, the threshold is lowered, therefore, the device is able to know when the user is relaxed.

The number of variations that are within the threshold divided by the variations that exceed the threshold is multiplied by the duration of the sample (5004), to allow the device to know the amount of time that the user has been mindfully relaxed. The sample can vary in length depending on the battery capacity of the device, and can be adjusted accordingly to reduce computational time to improve the battery performance of the device.

The device then begins to count up this time. When the user taps the ring again to end the meditation practice, the display on the ring indicates the percentage and amount of time the user has been mindful/relaxed during the session.

In the even that the wearer selects a focus-based meditation, the ring then adjusts the algorithm to calculate the user's concentration. As aforementioned in the background, focus meditations display the same signs in the Autonomic Nervous System as those of stress, however they are preceded by an intense relaxation.

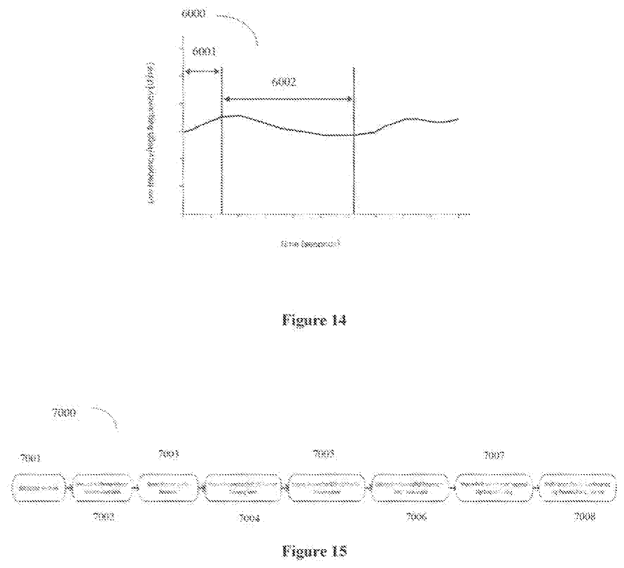

To mimic these circumstances, the device first informs the user to relax for a certain amount of time, which can be predetermined by the user. The device than indicates to the user when they are fully relaxed, after which point it then informs the user to start their focus meditation. To monitor the stress that the user is going through, the processor of the smart devices takes a sample of heart rate variability, i.e. the time interval between consecutive heart beats, and converts it into its equivalent frequency power spectrum using fast Fourier tranforms. From empirical evidence and literature, the frequency spectrum can be divided into a low frequency region (0.03-0.15 Hz) and a high frequency region (0.15-0.5 Hz), the ratio of the integrated power within the high frequency region over the integrated power of the low frequency region is referred to in literation as the LF/HF ratio, which indicates the Vagal Tone, or the sate of the Vagus nerve, an important nerve in the Autonomic Nervous System. A high ratio indicates an increased amount of stress and a lower one indicates a reduced stress. FIG. 14 illustrates graphical representation of the focus (6000), and highlights the changes in the ratio between low frequency and high frequency power of the user's heart rate variability during a focus meditation. The regions where the signal is rising (6001) indicate an effort of the user to remain stressed and hence, show them being focused, whereas the areas where the graph is decreasing (6002) indicates when no effort is applied any longer, and hence they are not being focused.

FIG. 15 shows flowchart illustrating method of calculation of mindful focus of a user during his/her meditation (7000). The device measures beat-by-beat heart rates of the user (7001) and measures the time difference between the consecutive heart beats (7002) thereby converting the time interval into frequencies (7003). The device then integrates the power from 0.03-0.15 Hz as low frequency power (7004); and the power from 0.15-0.5 Hz as high frequency power (7005). It further calculates LF/HF for every one minute of data (7006); and measure the time when LF/HF is rising (7007).

The smart wearable device then calculates the LF/HF ratio during the time the user has been relaxing to register a baseline. It then checks every consecutive sample in the focus meditation against the prior value to check if the user has been experiencing a sympathetic or a stressed state due to intense concentration. If the ratio begins to fall, it indicates that the user has been relaxing and not spending the time in concentration (7008).

Literature and empirical evidence suggest that the minimum duration for every sample is one minute, and hence the processing unit takes the amount of time the user has experiences stress and adds it up, which indicates the amount of time that the user has been in a state of focus mindfulness during their focus meditation.

The user can then tap the device again to inform that the meditation is at an end, at which point the device further indicates through the display the amount of time that the wearer has spent in a state of mindful focus.

In yet another embodiment of the present invention, the device sends this data collected, tracked, monitored and analyzed to any smart device namely mobile phone, computing device, laptop, tab, or to the cloud, allowing the user to view their data on the internet. Such connected devices such as mobile phones can track the number of minutes, and even send reminders to the user when the amount of time they have present into the mobile phone as their targeted number of minutes of mindfulness has not been reached.

1. A wearable smart device (100), with an in-built chargeable battery, in contact with a user for tracking the amount of time the user is mindful during a session of meditation comprising:
a. An input module allowing the user to select and register at-least one form of meditation session from a list consisting of breathing, relaxation and focus meditation to establish a session for monitoring;
b. A plurality of sensor modules recognising the input provided by the user;
c. A display module displaying the registered type of meditation that will be tracked by the device;
d. A plurality of sensor modules measuring beat-by-beat heart rate of the user;
e. A processing module (101), with an inbuilt wireless communication unit, to calculate the time difference between consecutive heartbeats and converting the same into frequencies in order to determine the mindfulness of the user during the session; and
f. A memory module (108), associated with the processor module, to store the collected and analysed data during the session.

2. The device as claimed in claim 1 wherein it measures other vital parameters of the user including heart rate variability, oxygen saturation, galvanic skin response, skin temperature and motion in 6-axis, and tracks user's sleep, exercise and stress level.

3. The device as claimed in claim 1 wherein the display module further includes communication modules to communicate with the user.

4. The device as claimed in claim 3 wherein the communication module includes notifying the user through RGB LEDs (105) and/or through vibrational motor.

5. The device as claimed in claim 1 wherein the plurality of sensor modules further includes photo plethysmograph (PPG) sensor in infra-red and red lights (102), 6-axis gyroscope (103), conduction sensor, touch detection sensor (104) and temperature sensor.

6. The device as claimed in claim 1 wherein it is further connected to another smart device(s) such as phone, tabs, computers, etc. through internet for archiving and analysis of the collected data.

7. The device as claimed in claim 5 wherein such other device locates and recommends the appropriate type of meditation for the user.

8. The device as claimed in claim 1 wherein the device is in the form of a ring (303) with a slit (302) that can be easily worn on any finger and further includes a strip of micro holes (502) on its surface letting the RGB LED (105) to shine through its enclosures.

9. The device as claimed in claim 7 wherein the device includes a means of charging through a handy charging dock (200), that is in power line communication (201) with the device (100) to send signal on the charge level of the device.

10. The device as claimed in claim 9 wherein the charging dock (200) includes a battery (201), battery charger (202) and a micro-controller (203).

11. The device as claimed in claim 9 wherein the charging dock (200) opens by sliding to reveal the device, and the device is automatically lifted up to make the device easily accessible.

12. The device as claimed in claim 1 wherein the input module includes receiving inputs from the user by way of swiping and/or tapping the surface/face of the device.

13. A method of monitoring a user's breathing during a session of meditation (3000) through the smart wearable device as claimed in claim 1, the method comprising the steps of:
a. Selecting and registering the breathing form of meditation to be monitored by swiping/tapping the display module of the device by user;
b. Tracking beat-by-beat heart rate of the user through PPO sensor module (3001) and calculating the difference between consecutive heartbeats by the processor module (3002);
c. Converting the detected intervals into frequencies by the processor module (3003);
d. Determining the frequency with higher power in the range of 0.03 to 0.5 Hz (3004);
c. Calculating the mindful breathing of the user by dividing the peak power obtained by the total power (3005);
f. Displaying the percentage and amount of time the user has been mindful in the session upon the user tapping the display module of the device to indicate the end of the session.

14. A method of monitoring a user's relaxation pattern during a session of meditation (5000) through the smart wearable device as claimed in claim 1, the method comprising the steps of:
a. Selecting and registering the relaxation form of meditation to be monitored by swiping/tapping the display module of the device by user;
b. Tracking beat-by-beat heart rate of the user through PPG sensor module (5001) and calculating the difference between consecutive heartbeats by the processor module (5002);
c. Checking variations, if any, in the detected intervals by the processor module (5003);
d. Calculating the mindful relaxation of the user by dividing the number of lower variations by total number of variations (5004); and
e. Displaying the percentage and amount of time the user has been mindful in the session upon the user tapping the display module of the device to indicate the end of the session.

15. A method of monitoring a user's focus during a session of meditation (7000) through the smart wearable device as claimed in claim 1, the method comprising the steps of:
a. Selecting and registering focus form of meditation to be monitored by swiping/tapping the display module of the device by user;
b. Tracking beat-by-beat heart rate of the user through PPG sensor module (7001) and calculating the time difference between consecutive heartbeats by the processor module (7002);
c. Converting: the detected intervals into frequencies by the processor module (7003);
d. Integrating the power in the range of 0.03-0.15 Hz as low frequency (LF) power (7004), and the power in the range of 0.15-0.5 Hz as high frequency (HF) power (7005);
e. Determining ratio of LF and HF (LF:HF) for every minute of data (7006), and the time when the LF:HF is rising (7007);
f. Calculating the mindful focus of the user by dividing the time when the LF:HF is rising by total time (7008); and
g. Displaying the percentage and amount of time the user has been mindful in the session upon the user tapping the display module of the device to indicate the end of the session.
